(19) United States
(12) Patent Application Publication
(10) Pub. No.: US 2013/0110203 A1
(43) Pub. Date: May 2, 2013

(54) Managing a Multi-function Coil in an Implantable Medical Device Using an Optical Switch

(71) Applicants: Vasily Dronov, San Jose, CA (US); Jordi Parramon, Valencia, CA (US); Daniel Aghassian, Glendale, CA (US); Md. Mizanur Rahman, Stevenson Ranch, CA (US); Emanuel Feldman, Simi Valley, CA (US)

(72) Inventors: Vasily Dronov, San Jose, CA (US); Jordi Parramon, Valencia, CA (US); Daniel Aghassian, Glendale, CA (US); Md. Mizanur Rahman, Stevenson Ranch, CA (US); Emanuel Feldman, Simi Valley, CA (US)

(73) Assignee: Boston Scientific Neuromodulation Corporation, Valencia, CA (US)

(21) Appl. No.: 13/611,518

(22) Filed: Sep. 12, 2012

Related U.S. Application Data
(60) Provisional application No. 61/552,362, filed on Oct. 27, 2011.

(51) Int. Cl. A61N 1/378

(52) U.S. Cl. 607/60

(57) ABSTRACT

Combination charging and telemetry circuit for use within an implantable medical device uses a single coil for both charging and telemetry that is controlled via the use of an opto-switch. One or more capacitors are used to tune the coil to different frequencies for receiving power from an external device and for the telemetry of information to and from an external device. The opto-switch is coupled to the resonant circuit, but because its input is electrically decoupled from its output, it easy to control.

### CROSS REFERENCE TO RELATED APPLICATIONS

This application is a non-provisional of U.S. Provisional Patent Application Ser. No. 61/552,362, filed Oct. 27, 2011, which is incorporated by reference and to which priority is claimed.

The present application is related to U.S. Pat. No. 8,155,752 (the '752 patent).

### FIELD OF THE INVENTION

This application relates to improved circuitry for an implantable medical device having a single coil for both telemetry and power reception.

### BACKGROUND

Implantable stimulation devices generate and deliver electrical stimuli to nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, occipital nerve stimulators to treat migraine headaches, and other neural stimulators to treat urinary incontinence, sleep apnea, shoulder subluxation, etc. The present invention may find applicability in all such applications and in other implantable medical device systems, although the description that follows will generally focus on the use of the invention in a Bion® microstimulator device system of the type disclosed in U.S. Patent Publ. No. 2010/0268309. However, the invention can also be used in a Spinal Cord Stimulator (SCS), such as is disclosed in U.S. Pat. No. 7,444,181, for example.

Microstimulator devices typically comprise a small, generally-cylindrical housing which carries electrodes for producing a desired stimulation current. Devices of this type are implanted proximate to the target tissue to allow the stimulation current to stimulate the target tissue to provide therapy for a wide variety of conditions and disorders. A microstimulator usually includes or carries stimulating electrodes intended to contact the patient's tissue, but may also have electrodes coupled to the body of the device via a lead or leads. A microstimulator may have two or more electrodes. Microstimulators benefit from simplicity. Because of their small size, the microstimulator can be directly implanted at a site requiring patient therapy.

FIG. 1 illustrates an exemplary implantable microstimulator 100. As shown, the microstimulator 100 includes a power source 145 such as a battery, a programmable memory 146, electrical circuitry 144, and a coil 147. These components are housed within a capsule 202, which is usually a thin, elongated cylinder, but may also be any other shape as determined by the structure of the desired target tissue, the method of implantation, the size and location of the power source 145, and/or the number and arrangement of external electrodes 142. In some embodiments, the volume of the capsule 202 is substantially equal to or less than three cubic centimeters.

The battery 145 supplies power to the various components within the microstimulator 100, such the electrical circuitry 144 and the coil 147. The battery 145 also provides power for therapeutic stimulation current sourced or sunk from the electrodes 142. The power source 145 may be a primary battery, a rechargeable battery, a capacitor, or any other suitable power source. Systems and methods for charging a rechargeable battery 145 will be described further below.

The coil 147 is configured to receive and/or emit a magnetic field that is used to communicate with, or receive power from, one or more external devices that support the implanted microstimulator 100, examples of which will be described below. Such communication and/or power transfer may be transcutaneous as is well known.

The programmable memory 146 is used at least in part for storing one or more sets of data, including electrical stimulation parameters that are safe and efficacious for a particular medical condition and/or for a particular patient. Electrical stimulation parameters control various parameters of the stimulation current applied to a target tissue including the frequency, pulse width, amplitude, burst pattern (e.g., burst on time and burst off time), duty cycle or burst repeat interval, ramp on time and ramp off time of the stimulation current, etc.

The illustrated microstimulator 100 includes electrodes 142-1 and 142-2 on the exterior of the capsule 202. The electrodes 142 may be disposed at either end of the capsule 202 as illustrated, or placed along the length of the capsule. There may also be more than two electrodes arranged in an array along the length of the capsule. One of the electrodes 142 may be designated as a stimulating electrode, with the other acting as an indifferent electrode (reference node) used to complete a stimulation circuit, producing monopolar stimulation. Or, one electrode may act as a cathode while the other acts as an anode, producing bipolar stimulation. Electrodes 142 may alternatively be located at the ends of short, flexible leads. The use of such leads permits, among other things, electrical stimulation to be directed to targeted tissue(s) a short distance from the surgical fixation of the bulk of the device 100.

The electrical circuitry 144 produces the electrical stimulation pulses that are delivered to the target nerve via the electrodes 142. The electrical circuitry 144 may include one or more microprocessors or microcontrollers configured to decode stimulation parameters from memory 146 and generate the corresponding stimulation pulses. The electrical circuitry 144 will generally also include other circuitry such as the current source circuitry, the transmission and receiver circuitry coupled to coil 147, electrode output capacitors, etc.

The external surfaces of the microstimulator 100 are preferably composed of biocompatible materials. For example, the capsule 202 may be made of glass, ceramic, metal, or any other material that provides a hermetic package that excludes water but permits passage of the magnetic fields used to transmit data and/or power. The electrodes 142 may be made of a noble or refractory metal or compound, such as platinum, iridium, tantalum, titanium, titanium nitride, niobium or alloys of any of these, to avoid corrosion or electrolysis which could damage the surrounding tissues and the device.

The microstimulator 100 may also include one or more infusion outlets 201, which facilitate the infusion of one or more drugs into the target tissue. Alternatively, catheters may be coupled to the infusion outlets 201 to deliver the drug therapy to target tissue some distance from the body of the microstimulator 100. If the microstimulator 100 is configured to provide a drug stimulation using infusion outlets 201, the microstimulator 100 may also include a pump 149 that is configured to store and dispense the one or more drugs.

Turning to FIG. 2, the microstimulator 100 is illustrated as implanted in a patient 150, and further shown are various external components that may be used to support the implanted microstimulator 100. An external controller 155 may be used to program and test the microstimulator 100 via communication link 156. Such link 156 is generally a two-way link, such that the microstimulator 100 can report its status or various other parameters to the external controller 155. Communication on link 156 occurs via magnetic inductive coupling. Thus, when data is to be sent from the external controller 155 to the microstimulator 100, a coil 158 in the external controller 155 is excited to produce a magnetic field that comprises the link 156, which magnetic field is detected at the coil 147 in the microstimulator. Likewise, when data is to be sent from the microstimulator 100 to the external controller 155, the coil 147 is excited to produce a magnetic field that comprises the link 156, which magnetic field is detected at the coil 158 in the external controller. Typically, the magnetic field is modulated, for example with Frequency Shift Keying (FSK) modulation or the like, to encode the data. For example, data telemetry via FSK can occur around a center frequency of 125 kHz, with a 129 kHz signal representing transmission of a logic ‘1’ and 121 kHz representing a logic ‘0’.

An external charger 151 provides power used to recharge the battery 145 (FIG. 1). Such power transfer occurs by energizing the coil 157 in the external charger 151, which produces a magnetic field comprising link 152. This magnetic field 152 energizes the coil 147 through the patient 150's tissue, and which is rectified, filtered, and used to recharge the battery 145. Link 152, like link 156, can be bidirectional to allow the microstimulator 100 to report status information back to the external charger 151. For example, once the circuitry 144 in the microstimulator 100 detects that the power source 145 is fully charged, the coil 147 can signal that fact back to the external charger 151 so that charging can cease. Charging can occur at convenient intervals for the patient 150, such as every night.

FIG. 3A shows the circuitry within microstimulator 100 that is coupled to coil 147. Such circuitry is explained in detail in the '752 patent that was incorporated by reference above. Therefore, the circuitry is only briefly explained here.

As explained in the '752 patent, the circuitry of FIG. 3A is beneficial because it uses a single coil L1 (147) receiving a magnetic charging field 152 from the external charger 151, and for transmitting and receiving data telemetry 156 to and from the external controller 155. (The external charger 151 and external controller 155 are shown in FIG. 3A as one integrated unit for simplicity).

Coil 147 is connected at one end through transistor switch M1 to a voltage, Vbat, provided by the battery 145 in the microstimulator 100, which may ranges from 2.5V to 4.2 Volts. Coil 147 is connected at its other end through transistor switch M2 to ground. Capacitor C1 is connected in parallel with coil 147, thus forming a resonant tank circuit, and tunes the tank circuit to a particular frequency for transmitting or receiving data telemetry to and from the external controller 155 (e.g., approximately 125 kHz). A series combination of a capacitor C2 and transistor switch M3 are also connected in parallel to coil 147. Transistor M3 is turned on during receipt of a magnetic charging field along link 152 from the external charger 151 to tune the tank circuit to the frequency of the magnetic charging field (e.g., approximately 80 kHz). Also connected in parallel with coil 147 is a full bridge rectifier represented by diodes D1-D4 for producing DC voltage Vout. A half bridge rectifier could also be used. Diodes D1-D4 may comprise, for example, Schottky diodes having forward voltage drops of approximately 0.4V. A transistor switch M4 is also connected between the rectifier circuitry and ground.

DC voltage Vout is received at storage capacitor C3, which smooths the voltage before being passed to charging circuitry 92. Charging circuitry 92 is used to charge battery source 145 in a controlled fashion. If needed, a Zener diode D5 or other suitable voltage clamp circuit may be connected across capacitor C3 to prevent Vout from exceeding some predetermined value, e.g., 6.2V.

FIG. 3B shows the status of transistor switches M1-M4 for the energy receive, data receive, and data transmit modes. As shown, to operate in an energy receive mode, the circuit will turn switches M1, M2 and M4 OFF, and will turn switch M3 ON. Turning M3 ON includes capacitor C2 in parallel with capacitor C1, which, in conjunction with the inductance formed by the coil 147, forms a resonant circuit which is tuned to the frequency of the magnetic charging field. The circuit of FIG. 3A may also operate in a data transmit mode during charging by employing back telemetry known as Load Shift Keying (LSK), in which case transistor M4 is modulated with the data to be transmitted back to the external charger 151. Switches M1-M4 is typically standard semiconductor switches, such as MOSFET switches.

For the circuit of FIG. 3A to operate in a data receive mode, the circuit will turn switches M1, M3 and M4 OFF, and will turn switch M2 ON. Turning M3 off excludes capacitor C2 from the resonant circuit, whose tuning is thus governed by coil 147 and capacitor C1. With capacitor C2 excluded, the resonant circuit is tuned to a higher frequency matching the operation of the external controller 155. Turning M2 ON grounds the resonant circuit, which provides an input to the receiver, which demodulates the received data (DATA RCV). The receiver can either comprise a differential input as illustrated in solid lines in FIG. 3A, or can comprise a single-ended non-differential input in which one of the inputs is grounded, as shown by the dashed line in FIG. 3A.

As further shown in FIG. 3B, the circuit of FIG. 3A may also operate in a data transmit mode by turning switches M3 and M4 OFF, by modulating switch M2 with a data signal (DATA XMIT), and by turning switch M1 ON. Under these conditions, the resonant circuit is once again, by virtue of transistor M3 being OFF, tuned to the higher frequency, and will broadcast a signal to the external control unit 151/155 along link 156 accordingly, with the energy for the radiation being supplied from the battery voltage, Vbat, via transistor M1. The transmitter receiving the data to be transmitted (DATA XMIT), is shown coupled to transistor M2, but could also couple to transistor M1.

Thus, it is seen that by selectively controlling the state of the switches M1-M4, the circuit of FIG. 3A may operate in different modes, using only a single coil 147. Such modes may be invoked in a time-multiplexed manner, e.g., with a first mode being followed by a second mode, depending upon the particular application at hand. Control signals M1-M4, as well as DATA XMIT, are ultimately issued by a microcontroller 160 in the microstimulator 100, and DATA RCV is received by that microcontroller.

The inventors have noticed that integrating such functionality into a circuit with only one coil 147 presents technical challenges. For one, it is difficult to operate a MOSEFT switch M1 during the energy receive mode because of the large swing in AC voltage produced at the drain of transistor M1 (node X in FIG. 3A). During the energy receive mode, transistors M1 and M2 are off, thus decoupling the coil 147 from Vbat and ground. As a result, the AC voltage across the coil 147 is somewhat indeterminate, but will still be bounded by the various diodes in the circuit. For example, the maximum voltage at node X will be determined by the threshold voltage of diode D3 and the breakdown voltage of Zener diode D5. Assuming low-threshold-voltage Schottky diodes in the rectifier (Vts=0.4V) and a breakdown voltage in the Zener diode D5 of 6.2V, node X will not exceed 6.6 Volts or so. At node Y, i.e., the drain of switch M2, the voltage is clamped by diode D2, such that node Y cannot drop below its threshold, i.e., −0.4V. Therefore, in accordance with the nature of the AC circuitry, the voltages at nodes X and Y will vary from −0.4V to 6.6V during the energy receive mode.

FIG. 3C shows switches M1 and M2 in cross section to better appreciate the problems of the prior art circuitry of FIG. 3A. Assuming switch M2 is an N-channel silicon MOSFET device as indicated by the p/n doped regions, there is no concern of leakage through that switch in the energy receive mode. The body diode B2 in switch M2 will have threshold voltage of about Vt=0.6V or so. Therefore, even at lower voltages present at node Y (i.e., −0.4 V), body diode B2 will not become forward biased and conduct; again, it is clamped and prevented from going below this voltage by Schottky diode D2. (It is assumed that the substrate of switch M2 is tied to its source and is thus held at ground, i.e., Vsub2=0V). Thus, there is no risk of inadvertent leakage from a traditional silicon MOSFET switch M2, and that transistor can be turned off as is desired in the energy receive mode.

By contrast, these same voltages at node X do present a leakage problem in switch M1. There is no means for clamping the voltage at node X akin to diode D2. Moreover, the substrate of switch M1 is typically connected to its source, Vbat (i.e., Vsub1=Vbat). As such, the body diode B1 in switch M1 will leak when the voltage at node X is less than Vbat−Vt. Given typical voltage ranges of 2.5V<Vbat<4.2V, body diode B1 will become forward biased when node X is at lower voltages, for example from −0.4V to perhaps 3V or so. This at least partially defeats the operation of switch M1, which is supposed to be off at all times during the energy receive mode. Worse, because the substrate of switch M1 is tied to Vbat, such leakage will occur directly from the battery 145. This is contrary to the desired purpose of the energy receive mode—which is to charge the battery−and also defeats the charging circuitry 92 (FIG. 3A), which is meant to isolate the battery during the energy receive mode and control its charging.

Better means of switching are possible in lieu of a traditional MOSFET switch M1, but would involve the use of multiple transistors, and more complicated means of control, such as the use of non-standard signals levels to control such transistors. Thus, a better solution is needed to support the sort of single-coil multi-function communication circuitry of FIG. 3A, and a solution is provided in this disclosure.

### BRIEF DESCRIPTION OF THE DRAWINGS

FIG. 1 shows a microstimulator of the prior art.

FIG. 2 shows a microstimulator of the prior art as implanted in a patient, as well as an external controller and an external charger.

FIGS. 3A-3C show the communication and charging circuitry in the microstimulator of the prior art, and the various modes in which such circuitry can be operated.

FIGS. 4A and 4B show the communication and charging circuitry in a microstimulator in accordance with an embodiment of the invention using an opto-switch, as well as the various modes in which such circuitry can be operated.

FIG. 5 shows further details of one example of an opto-switch useable in an embodiment of the invention.

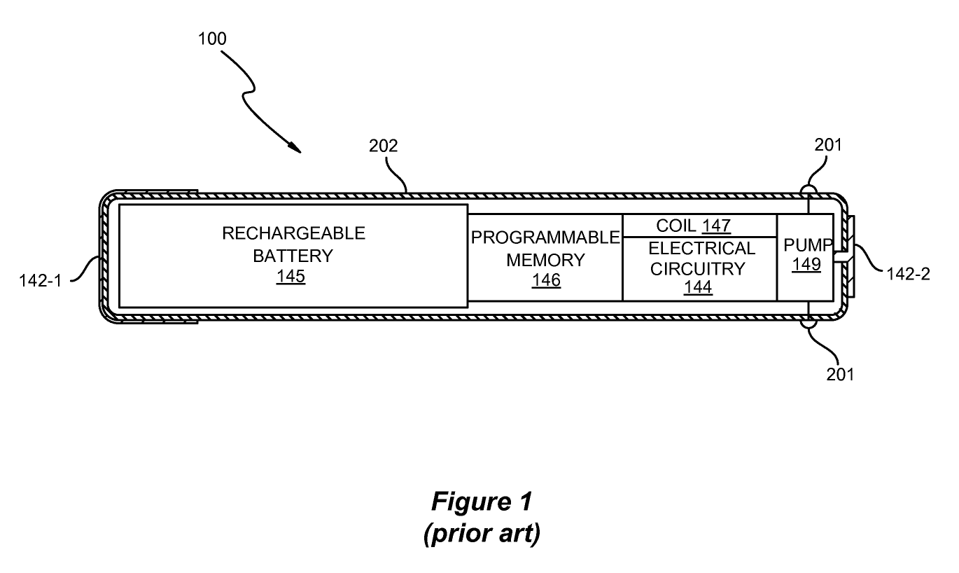

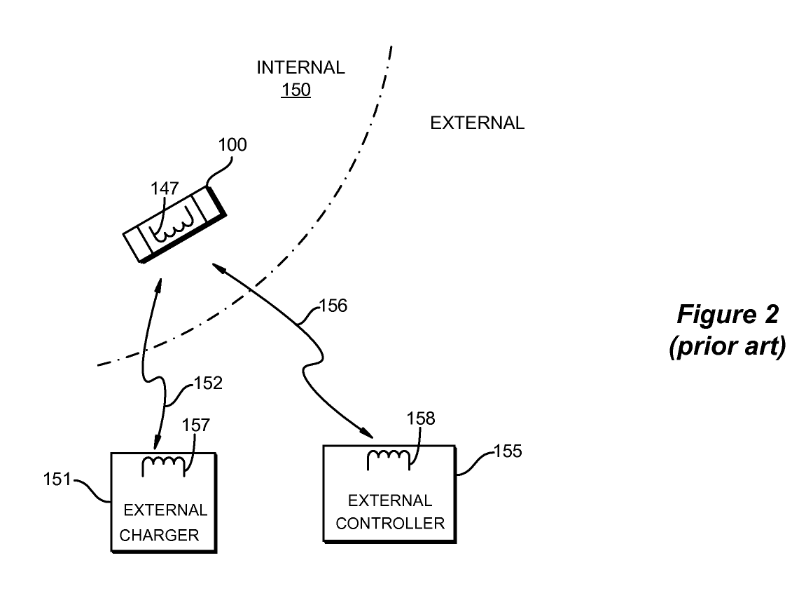

### DETAILED DESCRIPTION

Improved circuitry for an implantable medical device is disclosed that utilizes an “opto-switch” (i.e., an optically-isolated switch, such as a photocoupler) that allows for a single coil to be used safely and reliably for both charging and communication.

Opto-switches use light waves to provide switching control with electrical isolation between the photocoupler's input (its gating signal) and its output (the two terminal of the switch). An exemplary opto-switch contains a source of light (e.g., a near infrared light-emitting diode (LED)) controlled by an electrical gating control signal; an optical channel for transmitting that light; and a photosensor for receiving that light and shorting the two terminals of the switch. In other words, the gating control signal is optically coupled to the photosensor, thus controlling the opto-switch. The photosensor can be, for example, a photoresistor, a photodiode, a phototransistor, a silicon-controlled rectifier (SCR), or a triac. An opto-switch allows use of a low voltage gating control signal irrespective of the voltages at its output because the control signal is electrically decoupled from those output voltages.

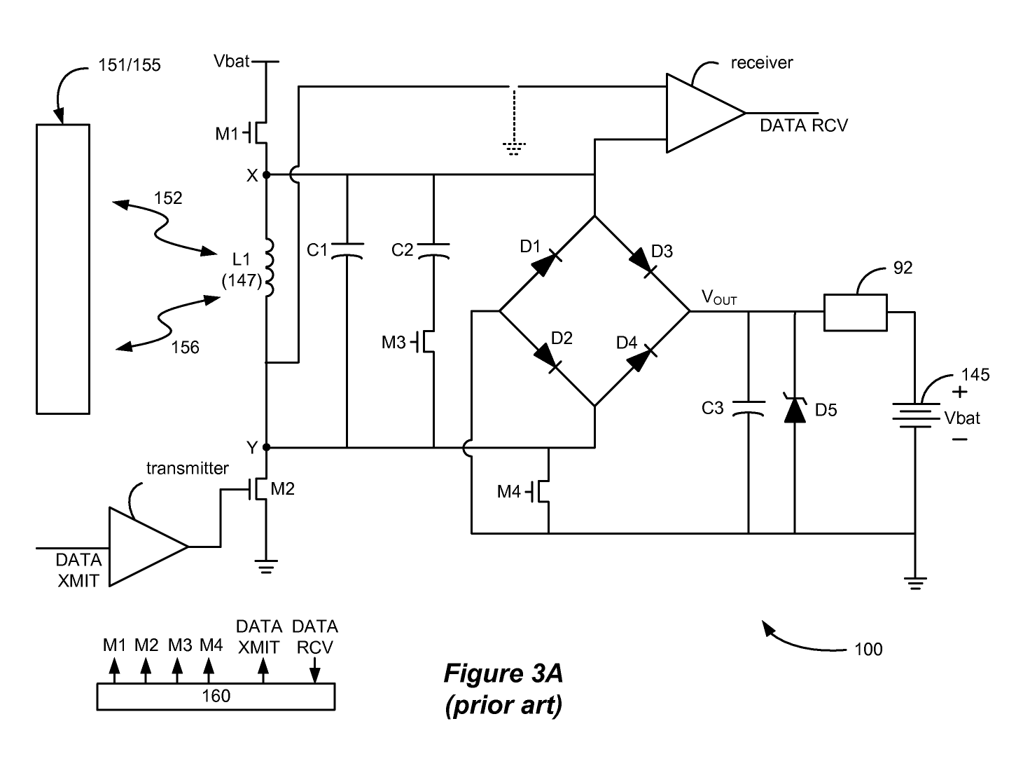

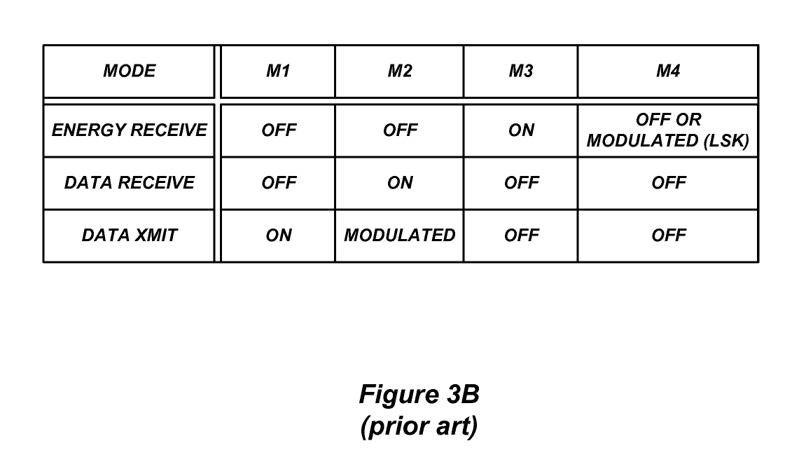

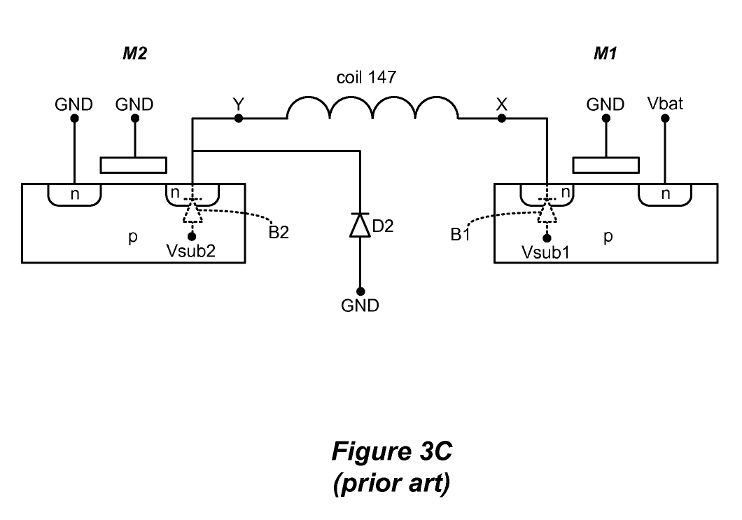

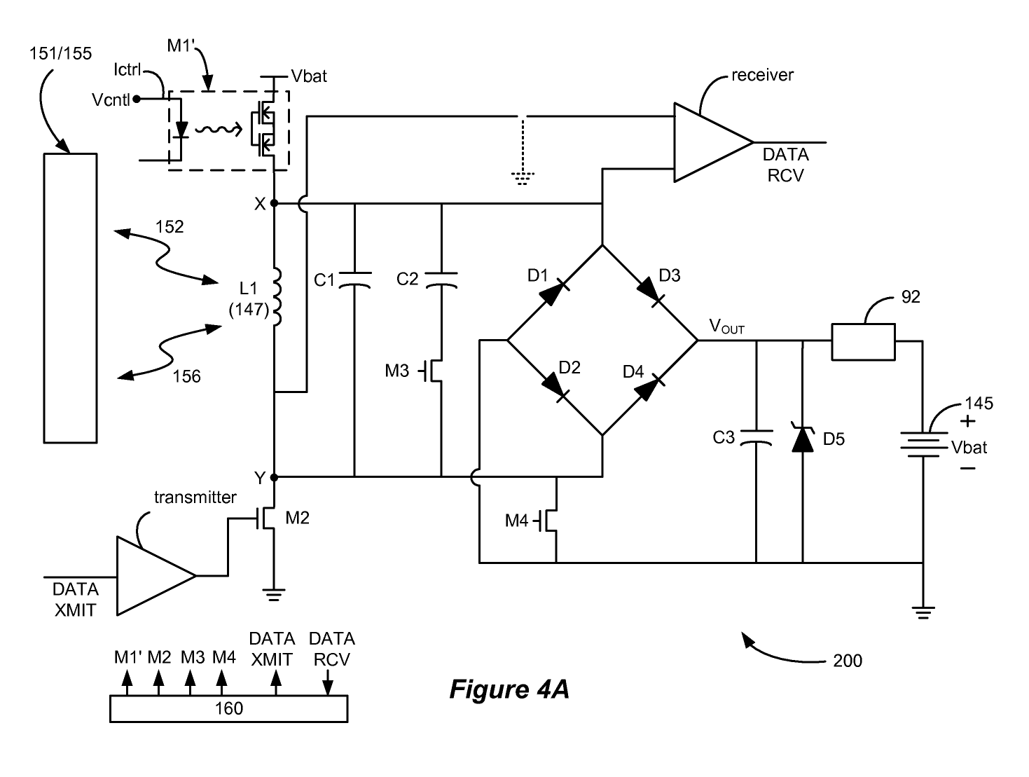

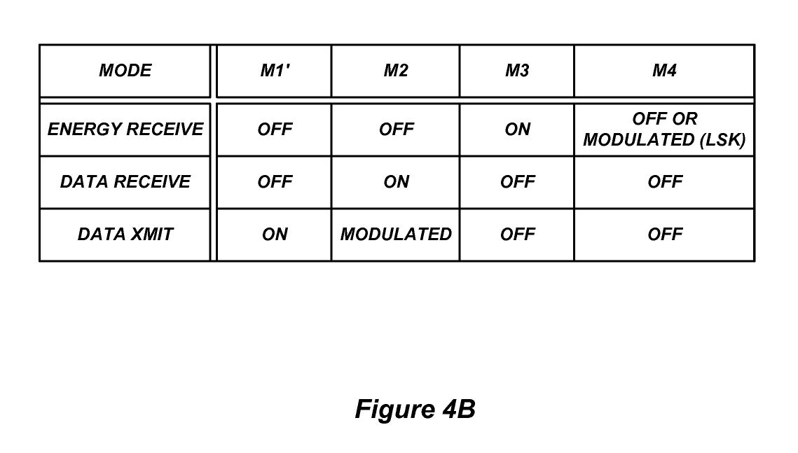

The inventors have realized that such a non-semiconductor-based switch is beneficial when gating AC signals in a resonant tank circuit, such as in the single-coil multi-function telemetry and energy receive circuitry of FIG. 3A, and such an improvement is shown in the improved microstimulator 200 of FIG. 4A. Similar to the single-coil circuitry of FIG. 3A, the single-coil circuitry of FIG. 4A can receive and transmit data, and can receive power for charging the implantable medical device. New to the circuitry of FIG. 4A is the use of an opto-switch M1′, which is controlled by gating control signal, Ictrl, which is electrically decoupled from the voltages on the output side of M1′, i.e., from Vbat and node X. As noted earlier, node X is subject to large voltage swings, particularly in the energy receive mode when M1′ is off. By electrically isolating node X from its gating control signal Icntl in the opto-switch M1′, this switch is easier to control. Moreover, the opto-switch M1′ is not subject to leakage regardless of the voltage level at node X.

FIG. 4B shows the status of switches M1′, M2, M3 and M4 when placing the improved microstimulator 200 in data transmit, data receive, and energy receive modes, and to tune the resonance of the tank circuit. These switches operate as before, including the opto-switch M1′, which is OFF during the energy receive mode, OFF during the data receive mode, and ON during the data transmit mode. Because the opto-switch M1′ is different from the MOSFET switches M2, M3, and M4, its gating control signal Ictrl may also differ, but is simple to generate. For example, it may only be necessary to provide a small voltage, Vctrl, at the input of the opto-switch M1′, which voltage is sufficient to forward bias the LED in the opto-switch and turn it on. A simple regulator circuit can be used to derive Vctrl if necessary. As before, the control signals for the switches can come from microcontroller 160. As the basic operation of the circuitry has not changed from its description in the Background, such operation is not repeated here.

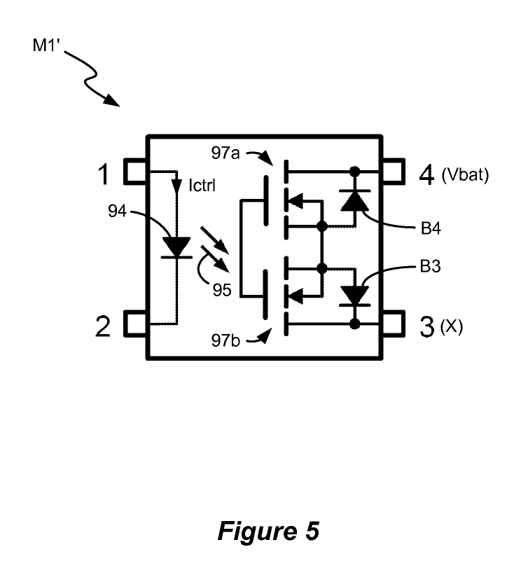

FIG. 5 shows the internal circuitry of an exemplary opto-switch that can be used for switch M1′, which can comprise for example TOSHIBA® Photocoupler Photo Relay, Toshiba Part No. TLP3231. This opto-switch is relatively small, measuring about 4.2×2.0×1.8 mm in volume, which is suitably small for inclusion inside an implantable medical device. Terminals 1 and 2 of opto-switch M1′ receive the gating control signal, Ictrl, which causes LED 94 to emit light 95 through the optical channel inside the switch.

This light is received by two serially-connected photo-sensitive MOSFET transistors 97a and 97b which act as the photosensor. When illuminated, these normally “off” transistors 97a and 97b are turned “on.” In other words, transistors 97a and 97b are normally open between Terminals 3 and 4, but become a short circuit when illuminated by the LED 94. These transistors 97a and 97b are thus completely electrically isolated from the gating control signal, Ictrl, making M1 switch easier to control even though subject to varying AC voltages at its output terminals.

Moreover, the two transistors in the opto-switch M1 are coupled “back to back,” resulting in two body diodes B3 and B4 which are back to back. Thus, and unlike the traditional MOSEFT switch M1 of the prior art (FIG. 3A), opto-switch M1′ cannot leak to the substrate: regardless of the voltage at node X, at least one of the body diodes B3 or B4 will be reversed biased, thus preventing leakage.

Modifications to the circuitry of FIG. 4A, and the opto-switch M1′, are possible. For example, it is not strictly necessary that the opto-switch M1′ occur on the high side of the coil 147 proximate to Vbat. With modification to the polarity of the circuit, the opto-switch could also occur on the low side of the circuit proximate to ground. Opto-switches could also be used in lieu of both of traditional MOSFET switches M1 and M2, although as explained earlier using an opto-switch for switch M2 is not necessary, at least in the context of the particular circuit of FIG. 4A. It is not strictly necessary that the transmitter be coupled to switch M2; it could also be coupled to the opto-switch M1′ at the high end of the circuit. Finally, while an opto-switch is particularly preferred to provide isolation between the switch's input and output, the mere use of back-to-back transistors could be used without optical coupling to its gating control input. That is, traditional electrical signals could be provided to the gates of the back-to-back transistors, although this may require the use of more complicated control signals. Still other modifications are possible.

Although illustrated as useful in the single-coil, multi-function communication circuitry of FIG. 4A, it should be noted that opto-switches can be used in connection with other types of communication circuitry present in an implantable medical device. In short, an opto-switch may be used in any such communication circuitry in which it is desirable to isolate AC voltages in the tank circuitry from the control for that circuit. One or more opto-switches may for example be used with a resonant tank circuit in which the coil and capacitor are serially connected.

While the invention herein disclosed has been described by means of specific embodiments and applications, numerous modifications and variations could be made thereto by those skilled in the art without departing from the literal or equivalent scope of the inventions set forth in the claims.

What is claimed is:

1. An implantable medical device, comprising:
a coil operable in at least two modes, wherein in a first mode the coil wirelessly receives power from an external device, and wherein in a second mode the coil wirelessly performs telemetry with an external device; and
a first switch, wherein an input to the first switch is optically coupled to and electrically isolated from an output of the first switch, and wherein switching between the first and second modes is controlled at least in part by the first switch.

2. The device of claim 1, wherein the coil receives power at a first frequency in the first mode, and wherein the coil performs telemetry at a second frequency in the second mode.

3. The device of claim 2, wherein the first frequency is non-modulated, and wherein the second frequency comprises a center frequency.

4. The device of claim 3, wherein the second frequency is modulated in accordance with a Frequency Shift Key (FSK) protocol.

5. The device of claim 1, wherein the first switch is off while the coil is operating in the first mode.

6. The device of claim 1, further comprising a first capacitor in parallel with the coil, wherein the first capacitor tunes the coil during the second mode.

7. The device of claim 6, further comprising a second capacitor, wherein the second capacitor is coupleable in parallel with the coil to tune the coil during the first mode.

8. The device of claim 1, wherein the first switch is connected to a first end of the coil.

9. The device of claim 8, wherein the first switch is connected between the first end of the coil and a first reference voltage in the implantable medical device.

10. The device of claim 9, further comprising a battery, and wherein the first reference voltage comprises a voltage of the battery.

11. The device of claim 9, further comprising a second switch between a second end of the coil and a second reference voltage in the implantable medical device.

12. The device of claim 11, wherein the second reference voltage is ground.

13. The device of claim 11, wherein the second switch is controlled by a transmitter operable during the second mode.

14. The device of claim 1, further comprising a rectifier coupled to the coil for producing a DC voltage during the first mode.

15. The device of claim 1, further comprising a receiver coupled to the coil operable during the second mode.

16. An implantable medical device, comprising:
a resonant circuit comprising an inductor and a first capacitor;
a first switch connected between a first end of the resonant circuit and a first reference voltage;
a second switch connected between a second end of the resonant circuit and a second reference voltage;
wherein an input to at least one of the first switch or the second switch is optically coupled to and electrically isolated from its output.

17. The device of claim 16, wherein the inductor and the first capacitor are wired in parallel.

18. The device of claim 16, further comprising a series connection of a second capacitor and a third switch, wherein the series connection is connected in parallel with the resonant circuit.

19. The device of claim 18, wherein the third switch is controllable to control a resonant frequency of the resonant circuit.

20. The device of claim 16, further comprising a rectifier in parallel with the resonant circuit.

21. The device of claim 16, further comprising a transmitter, wherein the transmitter controls either the first or second switch.

22. The device of claim 21, wherein the other of the first or second switch not controlled by the transmitter is on when the transmitter is operating.

23. The device of claim 16, wherein the first and second switches are off when the resonant circuit is wirelessly receiving energy from an external device.

24. The device of claim 16, further comprising a receiver coupled to the resonant circuit.

25. The device of claim 24, wherein the receiver is coupled in parallel with the resonant circuit.

26. The device of claim 24, wherein only one of the first and second switches are on when the receiver is operating.

27. The device of claim 16, wherein the first reference voltage comprises a power supply voltage, and wherein the second reference voltage comprises ground.

28. The device of claim 27, further comprising a battery, and wherein the power supply voltage comprises a voltage of the battery.
